(19) United States
(12) Patent Application Publication
(10) Pub. No.: US 2011/0245095 A1
(43) Pub. Date: Oct. 6, 2011

(54) FAST ASSIGNMENT OF ADEQUATE CHEMOTHERAPY WITH LATINUM BASED DRUGS FOR CANCER PATIENTS BASED ON THE IDENTIFICATION OF CONSTITUTIONAL BRCAL MUTATIONS

(76) Inventors: Tomasz Byrski, Szczecin (PL); Jacek Gronwald, Szczecin (PL); Jan Lubinski, Szczecin (PL)

(21) Appl. No.: 12/996,827

(22) PCT Filed: Jul. 6, 2009
(86) PCT No.: PCT/PL2009/050011

§ 371 Date: Jun. 17, 2011

(30) Foreign Application Priority Data
Jul. 6, 2008 (PL) PL385589

(51) Int. Cl. C40B 30/04; C12Q 1/68; C40B 30/00; C40B 40/06

(52) U.S. Cl. 506/9; 435/6.13; 435/6.12; 506/7; 506/16

(57) ABSTRACT

A new method to improve therapy outcome depending on a particular constitutional genotype have been disclosed. Subject of invention allows to synthesize DNA and identification of germline BRCA1 genetic abnormalities which are correlated with a significantly increased clinical response to chemotherapy based on platinum derived drugs in cancer patients.

### FIELD OF THE INVENTION

Mode and composition for optimizing the efficiency of chemotherapy, depending on the particular constitutional genetic profile in each cancer patient. Generally, the invention concerns a new method to improve therapy outcome depending on a particular constitutional genotype. Subject of invention allows to synthesize DNA and identification of germline BRCA1 genetic abnormalities which are correlated with a significantly increased clinical response to chemotherapy based on platinum derived drugs in cancer patients.

### BACKGROUND OF THE INVENTION

Constitutional mutations in the gene BRCA1 are the main factor responsible for high risk monogenic predisposition to breast and ovarian cancer (Ford et al. Am. J. Human Genet. 1998, 62:676-89; Narod et al. Am. J. Hum. Genet. 1995, 56:254-64; Narod et al. Am. J. Hum. Genet. 1995, 57:957-8). Biological effects of BRCA1 belong to a reduced group of situations, where distinct abnormalities of just one protein lead to very critical consequences, almost independently of modifiers and environmental factors, as evidenced here by the very high risk of breast and ovarian cancer risk of BRCA1 mutation carriers throughout different human populations. To date hundreds of constitutional mutations have been described for BRCA1 (BIC database). Some of these are recurrent mutations with founder effect, i.e. show a population-specific profile. For example, and without loss of generality, there are founder mutations characteristic for the Ashkenazi Jewish (Tonin et al. Nat Medicine 1996; 2:1179-83), Finnish (Huusko et al. Am J Hum Genet. 1998; 62:1544-8), Danish (Bergthorsson et al. 1 Med Genet. 2001; 38:361-8), Italian (Russo et al. Breast Cancer Res Treat 2007, 105(3):267-76), English (Anglian Breast Cancer Study Group Br J Cancer 2000, 83(10):1301-8), Portuguese (Pexoto et al. Fam Cancer 2006, 5:379-87), Indian (Hedau et al. Breast Cancer Res Treat 2004, 88:177-86; Saxena et al. BMC Med Genet. 2006, 7:75; Valarmathi et al. Hum Mutat 2004; 23:205), Japanese (Ikeda et al. Int J Cancer 2001, 91:83-8), Turkish (Yazici et al. Br J Cancer 2000, 83:737-42), Pakistani (Rashid et al, Int J Cancer 2006, 119:2832-9), Korean (Seo et al. Hum Mutat 2004, 24:350; Han et al. Clin Genet. 2006, 70:496-501), Dutch (Peelen et al. Am J Hum Genet. 1997; 60:1041-9; Petrij-Bosch et al. Nat Genet. 1997, 17:341-5) or Canadians of French (Tonin et al. Am J Hum Genet. 1998, 63:1341-51) and Canadians of English origin (Risch et al. Am J Hum Genet. 2001, 68:700-10) among many others.

Analogous founder mutations can also be found in Slavic populations, as shared by e.g. the Polish (Gorski et al. Am J Hum Genet. 2000; 66:1963-8), the Chech (Machackova et al. Cas Lek Cesk 2000; 139:635-7), the Latvian (Csokay et al. Hum Mutat 1999; 14:92), the Belorussian (Oszurek et al. Clin Genet. 2001; 60:470-1), or the Russian (Tereschenko et al. Hum Mutat 2002; 19:184). The Polish patent application nr P. 335 917 describes founder mutations of the gene BRCA1 characteristic for the Slavic population. Another Polish patent application with nr P. 364 413 shows that ˜90% of all BRCA1 mutations in Poland belong to one out of three common mutations: BRCA1 exon 20 5382insC, BRCA1 exon 5 G61G and BRCA1 exon 11 4153delA.

In summary, we can conclude that the current state of the art shows a strong correlation between germline mutations in the gene BRCA1 and predisposition to breast and ovarian cancer, whereas the incidence of each particular mutation is different in different ethnic groups. Subject of this invention is a method for predicting response to chemotherapy in cancer patients, who have already developed a tumor, dependent on their constitutional BRCA1 genotype.

Cisplatin, cis-PtCl₂(NH₃)₂, is known since 1845 when it was first synthesized by M. Peyrone (Ann Chemie Pharm 1845; 51:129). In 1965 Rosenberg et al. discovered that platinum derivatives were able to inhibit cell division in the bacteria Escherichia coli (Nature 1965, 205(4972): 698-699). They developed a series of experiments to test the effects of different platinum complexes on sarcomas artificially implanted in rats. That study found that cis-diamminedichloridoplatinum(II) was the most effective platinum complex eliminating those sarcomas. Human trials produced positive results, limited, to some extent by toxic side effects. Once the side effects could be made bearable through the use of adjuvant therapies, the compound's effectiveness on cancer patients was proven. In 1978 it was finally approved by the United States Food and Drug Administration for treatment of certain cancers.

Cisplatin binds irreversibly to DNA and interferes with its repair mechanism, eventually leading to cell death. Following administration, one of the chloride ligands is slowly displaced by water, in a process termed aquation. The resulting structure can then bind to a single nitrogen on a DNA nucleotide. Then, the second chloride is replaced by another H2O and the platinum binds to a second nucleotide. The platinum cross-links two bases via displacement of the other chloride ligand, and thus interferes with mitotic cell division. The cisplatin-DNA complex is identified as DNA error by the HMG (high mobility group) and other DNA repair proteins which become irreversibly bound. However the resulting distortion to the shape of the DNA prevents effective repair, and apoptosis or programmed cell death gets activated as repair proves impossible (Jamieson & Lippard; Chemical Reviews 1999; 99(9): 2467-2498; Bruhn et al. Prog Inorg Chem: Bioinorg Chem 1991; 38: 478-517). Other antineoplastic agents, such as etoposide, contribute to the platinum-DNA-protein complex and thus synergistically reinforce the activity of cisplatin. Such synergistic effects are also found with several other anticancer drugs such us 5-fluorouracil, cytarabine and bleomycin.

Cisplatin is currently widely prescribed for a variety of tumors (germ-cell, bladder carcinoma, adrenal cortex carcinoma, breast cancer, head and neck carcinoma, osteogenic sarcoma, Hodgkins lymphoma, prostate carcinoma, cervix carcinoma, esophageal carcinoma or lung carcinoma, among others). Although active in many tumors, state of the art is that cisplatin is regularly curative in only one, testicular (Ebbing et al. Aktuelle Urol. 2008, 39(6):429-35).

One of the significant limitations towards the successful treatment of malignancies with cisplatin and other platinum-based drugs is the emergence of drug resistant tumour cells, Studies on ovarian carcinoma cells resistant to cisplatin have shown modified DNA for most of the twenty six chromosomes. These changes were typically gene excision and insertion, which suggest that the acquired resistance to the drug may be associated with substantial genomic instability. The consequences of such wide genetic alteration would explain the occurrence of the many mechanisms observed for cisplatin resistance. One important mechanism involves the inactivation of a cisplatin-dependent p53-accumulation pathway. P53 is the primary regulatory protein that initiates repair mechanisms when it identifies altered DNA and induces apoptosis if the repair process is not successful. It is suggested that drug resistance is not related to the amount of cross-links formed, but partially due to this inactivation. The consequence of this is reduced activation of DNA-damage repair mechanisms which would ultimately lead to an inefficient induction of apoptosis mechanisms and, paradoxically, would preserve cell's life (Stordal & Davey IUBMB Life 2007; 59(11): 696-699).

A second drawback for the clinical use of cisplatin are the serious and mostly irreversible side effects associated, like nephrotoxicity (renal damage), neurotoxicity (nerve damage), emesis (nausea and vomiting), ototoxicity (hearing loss), alopecia (hair loss), myelosupression (reduction of bone marrow function) and disturbance of the electrolyte balance, along with many other side effects which are comparatively minor. The major dose-limiting effect is nephrotoxicity. It is dose-dependent, irreversible in some cases, and primarily affects the proximal tubules. Renal damage caused by cisplatin includes tubular degeneration, loss of brush border, necrosis and mineralisation of tubular epithelial cells, all of which are cumulative. Damage to the kidneys can be minimized through the administration of continuous IV hydration along with diuretic drugs before and following the infusion of cisplatin (Leggha et al. J. Clin. Oncol. 1985, 3(10):1373-8)

A final toxicological aspect of cisplatin is its presumed carcinogenicity (ability to cause cancer) and mutagenicity (ability to alter DNA). Many studies have indicated these effects in animal models or cell lines (Lin et al. J. Inorg. Biochem. 1999, 77(1-2):89-93).

Cisplatin, although a very potent and successful antineoplastic, is also very toxic. Therefore intensive work has been performed to develop more efficient new-generation platinum derivatives with reduced side effects and resistance:
Carboplatin is a chemotherapy drug mainly used against ovarian carcinoma, lung, head and neck cancers. It has since gained popularity in clinical treatment due to its vastly reduced side-effects compared to cisplatin, particularly the elimination of cisplatin's nephrotoxic effects. Nausea and vomiting are less severe and more easily controlled. The main drawback of carboplatin is its myelosuppressive effect.
Oxaliplatin is a platinum-based chemotherapy drug typically administered in combination with antibiotics for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Side-effects of oxaliplatin are clearly less severe than carboplatin or cisplatin for ototoxicity and nephrotoxicity. However other side-effects are still very relevant, such as neuropathy, fatigue, nausea, vomiting, diarrhea and neutropenia.
Triplatin tetranitrate is a new platinum-based cytotoxic drug for the treatment of human cancer. It is a trinuclear platinum coordination complex, with chloride and amine ligands. The drug acts by forming coordinate covalent adducts with cellular DNA, preventing DNA transcription and replication, and through this inducing apoptosis. It is structurally similar to, but in a different family from, the anticancer drugs cisplatin, carboplatin and oxaliplatin. The maximum tolerated dose is clearly lower than other platinum-based drugs, side-effects being largely severe diarrhea, cramps and vomit.
Nedaplatin is a platinum compound which produces less nausea, vomiting and nephrotoxicity than cisplatin.
Satraplatin is the first platinum compound that is orally active, in contrast to all other platinum-based drugs, where dosage is intravenous. It is currently under investigation as an alternative treatment for patients with prostate cancer who have failed previous chemotherapy.

The clinical application of platinum derived chemotherapeutic drugs in the field of oncology has been object of several patents for its independent use (e.g. WO2008051525, US2008069901, US2007254045) or in combination with other drugs (e.g. US2008166344, US2006281736, WO2008077772). Thus, both platinum derived chemotherapeutic drugs and their application will be considered as conventional from now on.

It is evidenced that the current use of platinum derivatives for chemotherapy against cancer is associated with severe side-effects. It is also evidenced that therapeutic efficiency of those drugs is highly variable in different cancer patients. As can be deduced from the foregoing state of the art, an objective problem is the lack of a method that could reliably classify patients in groups of responders and non-responders towards platinum based drugs before therapy onset to reduce the time needed to find the adequate drug, thus improving therapy success and reducing the impact of unwanted side-effects.

First methods have already been reported that predict the outcome of platinum based therapy, focusing on the expression levels of several genetic markers such as PDE3B (phosphodiesterase 3B), PDGFC (platelet derived growth factor C), PKD2 (polycystic kidney disease 2), NRG1 (neuregulin 1) and LUM (lumican) as reported in WO2008047947, as well as p63 and p73 isoforms as reported in WO2008045344, or even BRCA1 as is subject of patents WO2004042080 and WO2005121786.

In the latter, basing on in vitro studies, it is suggested that breast cancer cell lines with low BRCA1 activity are not responsive to chemotherapy with taxanes, and make thus recommendable to choose a DNA damaging agent, such as platinum derivatives, instead. Under the possible ways to determine a reduced BRCA1 activity, it is suggested the analysis of somatic mutations in the gene BRCA1 in breast tumor biopsy material (not constitutional mutations).

However, for the use of that method in clinical practice, some problems arise. A major objection is the generalization of the in vitro model to a human subject. The development of the tumor is influenced by many factors, such as permeability to the tumor cells, interstitial hypertension, metabolic degradation, immune response or angiogenesis among others, that greatly diverge between in vivo and in vitro studies and most remarkably the context of metabolites taken to and from the tumor site by blood circulation, e.g. regulator molecules expressed elsewhere, is completely absent in vitro. These divergences often account for a lack of correlation of the effect of anticancer drugs in vivo and vitro (Williams et al. Cancer Res 2000; 60:6045-51; Poondru et al. Invest New Drugs 2002; 20:23-33, McCready et al. J. Natl. Cancer Inst 1989; 81: 682-7).

But most importantly, whenever chemotherapy is advisable and most particularly for neoadjuvant chemotherapy, there is an immediate urge from the clinical point of view in determining the most efficient type chemotherapy for the patient. In this scenario, a method is needed that allows a fast decision-making for the oncologist. The methods described in WO2004042080 and WO2005121786 do not fulfill these requirements, since they require biopsy material and check for the presence of somatic mutations, de novo mutations, which has necessarily to rely on a time and cost intensive technique such as is whole-gene sequencing.

### SUBJECT OF THE INVENTION

The subject of the present invention is a method for predicting response to platinum based chemotherapy of a cancer patient, depending on his constitutional BRCA1 genotype, characterized by analysis of any genetic material obtained from the patient. In fact, as the method is focused on germline founder mutations, the prediction of the response to a possible future therapy is already possible at a stage where the individual is just identified as predisposed to cancer in the frame of a standard genetic scan for cancer associated markers, as is often performed e.g. in families with high cancer incidence or for family members of BRCA1 mutation carriers even in absence of family cancer aggregation. Thus, before he develops a cancer.

As mutations of the BRCA1 gene it is understood mutations affecting the genetic sequence of the gene BRCA1, as well as flanking mutations in the direct neighborhood of BRCA1, which are classified for example in the database of the Breast Cancer Information Core (BIC), without loss of generality. The database is available in the internet under http://research.nhgri.nih.gov/bic/. The numeration system of the genetic sequence and the terminology to denominate the mutations used in the current patent comply with the established scientific terminology in this area. As founder mutations, it is understood those among the aforementioned ones, which appear with a characteristically high frequency in specific human populations with a common ethnic origin.

In the context of this invention, the patient is ideally characterized, without loss of generality, as a patient of known ethnic origin. As a first example, a patient of Slavic origin may be scanned with an ethno-specific founder mutation panel, where the main founder mutations of BRCA1 gene observed in that population are 5382insC, 300T→G and 4153delA. As a second example, a patient of Ashkenazi Jewish origin may be scanned with an ethno-specific founder mutation panel, where the main founder mutations of BRCA1 gene observed in that population are 185delAG and 5382insC. Exemplary, without loss of generality, a sample founder mutations characterizing different ethnic populations is summarized in table 1.

TABLE 1
Sample of BRCA1 founder mutations

| Founder BRCA1 | |
|---|---|
| mutations | Ethnic populations |
| S1503X | Pakistani |
| R1835X | Pakistani |
| 185insA | Pakistani |
| 185delAG | Ashkenazi Jewish, Hungarian, Indian, |
| | Hispanic, Pakistani |
| 5382insC | Ashkenazi Jewish, Turkish, Slavic, Hungarian |
| 300T→G | Slavic, Hungarian |
| 4153delA | Slavic |
| 3171ins5 (3166ins5) | Scandinavian |
| 2595delA | Scandinavian |
| 1806C→T | Scandinavian |
| 1201del11 | Scandinavian |
| 1135insA | Scandinavian |
| 1675delA | Scandinavian |
| 2804delAA | Dutch |
| Alu-mediated deletions | Dutch |
| ex13, ex22 | |
| 4446C→T | French Canadian |
| 2953del3 + C | French Canadian |
| 3300delA | Thai |
| Asp67Glu | Thai |
| 2156delinsCC | Portuguese |
| 3450del4 | Portuguese |
| 2552delC | Hispanic |
| R1443X | Hispanic |
| S955X | Hispanic |
| IVS5 + 1G > A | Hispanic |
| Tyr978X | Non-Ashkenazi Jews |
| 943ins10 | West-African |
| IVS13 + 1G > A | West-African |

A genetic analysis of BRCA1 germline mutations based on population-specific panels of known founder mutations is particularly favorable, since it allows a highly reliable identification of the most common alterations in BRCA1 with conventional indirect techniques based on DNA or RNA within a question of hours. The mutations may he detected directly or indirectly at DNA. RNA or protein level, but particularly favorable in the context of this invention is the analysis of DNA or RNA for the indirect identification of mutations with one of the following techniques: ASO PCR (allele specific-polymerase chain reaction), SSCP (single-strand conformation polymorphism), ASA (allele specific analysis), RFLP-PCR (restriction fragment length polymorphism—polymerase chain reaction), Taqman RT-PCR (real-time PCR), mass spectrometry (e.g. MALDI-TOF) or microarray technology. Examples of primers that can be used for the amplification of such sequences of the gene BRCA1 in the context of the present invention are presented in tables 2 and 3.

Analogously, it is also favorable the genetic analysis of BRCA1 germline mutations based on a larger, unspecific panel comprising all known BRCA1 constitutional mutations, or a sample of the most frequent ones, to be applied for patients with unknown or mixed ethnic origin.

In the context of this invention, the biological material subject of genetic analysis is not necessarily a tumor biopsy. In the contrary, somatic changes are difficult to identify and mostly require time-consuming direct DNA or RNA sequencing techniques since, unlike constitutional mutations, they may occur at any position of the sequence. It is of critical relevance in clinical practice to assign the correct chemotherapy, whenever needed, as soon as possible. Thus, the identification of the constitutional BRCA1 genotype should be preferably performed on biological material as easily available as possible, such as peripheral blood or saliva. This is a clear advantage in comparison with tumor biopsies, where the access to tumor material is more difficult and sometimes impossible. Most important, the analysis of somatic mutations has to rely necessarily on full gene sequencing, which implies high costs and most critical: time delay. The analysis based on constitutional BRCA1 mutations constrains the spectrum of potential therapies already from the outset, even before development of a tumor, just basing on the genetic profile of the individual.

In the context of this invention, platinum based DNA damaging drugs comprise cisplatin, carboplatin, oxaliplatin, triplatin, nedaplatin, satraplatin and their known derivatives and analogues. Treated tumors comprise not only malignancies known from literature to occur with increased probability among BRCA1 mutation carriers such as prostate cancer, leukemia, lymphoma and particularly breast and ovarian cancer. But, as revealed from our experiments, also other types of cancer such as cancer of the colon, esophagus, pancreas, gallbladder or lung. To date all seven treated cancer types in our institution (see examples) respond to platinum based therapy, whenever the patient has a constitutional BRCA1 mutation. Therefore, in the context of this invention, treated tumors include virtually all types of cancer. In the context of this invention BRCA1 mutations are constitutional mutations.

The invention is described in the following examples of application, to better illustrate its relevance. However, the invention cannot be reduced to the mentioned examples.

### EXAMPLE 1

Increased Response to Neoadjuvant Cisplatin Therapy in Breast Cancer Patients, which Carry a Constitutional Mutation in the Gene BRCA1.

Chemotherapy for breast cancer is often given prior to primary surgery (neoadjuvant chemotherapy) to decrease the size of the tumor in order that breast-conserving surgery can be performed, and to reduce the probability of lymph node involvement (Gralow et al. J. Clin. Oncol, 2008, 26:814-9). A further advantage of neoadjuvant therapy is that it helps to assess chemosensitivity to a particular agent; i.e. if the tumor does not diminish in size, then it is unlikely that the treatment will benefit the patient. In some cases, the breast cancer will regress completely, with no evidence of residual tumor, either in breast or the axilla (pathological complete response). In longitudinal studies, pathological complete response is predictive of prolonged disease-free survival and of overall survival (Liedtke et al. J. Clin. Oncol. 2008, 10: 1275-81).

The majority of BRCA1 mutation carriers diagnosed with breast cancer receive chemotherapy, because their tumors are typically high-grade (Lakhani et al. J. Natl. Cancer Inst. 1998, 90:1138-45), are estrogen-receptor negative (Foulkes et al. Clin. Cancer Res. 2004, 10: 2029-34) and exhibit rapid growth (Tilanus-Linthorst et al. Clin. Cancer Res. 2007, 13: 7357-62). In two historical cohort studies, BRCA1-associated breast cancers appeared to be more sensitive to various forms of chemotherapy than non-hereditary tumors (Robson et al. Breast Cancer Res. 2004, 6:R8-17; Rennert et al. N. Engl. J. Med. 2007, 357:115-23). There is increasing evidence that the DNA-repair defects, which are characteristic of BRCA1-related cancers, confer sensitivity to particular systemic agents. For example, in vitro studies of BRCA1-associated breast cancer cell lines show a high degree of sensitivity to agents that bring about inter-strand cross-links, such as platinum-based drugs (Chabalier et al. Cell Cycle 2003, 5:1001-7; Kennedy et al. J. Natl. Cancer Inst. 2004, 96:1659-68; Zhou et al. Oncogene 2003, 22:2396-40).

Platinum-based compounds have not been found to benefit the majority of breast cancer patients (Decatris Cancer Treatment Reviews 2004; 4 53-81). The objective of this study was to assess the frequency of complete pathologic response following neo-adjuvant cisplatin chemotherapy in women with breast cancer and a BRCA1 mutation.

### Study Design and Eligibility Criteria

Patients were eligible for the study if they had a pathologically-proven diagnosis of invasive breast cancer by core biopsy or fine-needle aspiration biopsy and if they carried a pathogenic BRCA1 mutation. Eight patients were treated in Szczecin (Poland) and two were treated in Bielsko-Biala (Poland). In these centers, genetic testing is offered to all women with incident breast cancer.

For the purpose of this study, genetic testing was expedited; in most cases the test result was available within five days of diagnosis. Written informed consent was obtained from each patient. The protocol was approved by the Research Ethics Review Board of the Pomeranian Medical Academy.

Patients with a previous diagnosis of breast cancer, or other cancer, were excluded (three patients). Patients who received prior chemotherapy were excluded (three patients). Patients with metastatic disease (stage 1V) at presentation were excluded (one patient). One patient presented with primary breast cancer after a previous prophylactic bilateral mastectomy and was excluded.

No patient received any medication that affected renal function; none was pregnant or breastfeeding, and none had an active infection, symptomatic congestive heart failure, unstable angina pectoris, cardiac arrhythmia, or psychiatric illness. Patients were evaluated for renal function, liver function, and hematology prior to therapy. All were within normal limits.

Prior to platinum chemotherapy, the clinical and histological features of the breast cancers were assessed. Tumor size was evaluated by clinical examination and by ultrasound, in some cases supplemented by mammogram. Histology was evaluated in nine cases by core biopsy and in one case by fine needle aspiration. For the nine cases who underwent core biopsy, grade, ER, PR, and ERBB2 status were evaluated by immuno-histochemistry. Immunohistochemical studies were not done for the patient who had a fine needle biopsy. Grade was evaluated in the core biopsy specimen using the Bloom-Richardson classification system,

### Genetic Testing

BRCA1 testing was conducted for the three founder mutations seen in Poland, namely C61G, 5382insC and 4153delA. A mutation analysis for mutations 4153delA and 5328insC was carried out by a multiplex allele-specific polymerase chain reaction (PCR) assay. The third common mutation (C61G) was detected with the help of a restriction enzyme site in exon 5 specific for that mutation. All patients could be successfully genotyped.

Identification of several mutations may be carried out grouped or independently. In the former case the primers set comprises an oligonucleotide pair for the identification of the mutation and a second oligonucleotide pair for control (table 1). However, it is particularly favorable the use of primer sets for a single multiplex PCR reaction (table 2).

TABLE 1
Primer sets for analysis of BRCA1 mutations with conventional PCR.

| Primer | | | Primer for sense strand | Primer for antisense strand |
|---|---|---|---|---|
| pairs | Primer ID | Function | [F] 5′->3′ | [R] 5′->3′ |
| Pair 1 for | B1-5382INSCI1 | identification | CAC TTC CAT TGA | TAC CTT TCT GTC CTG |
| BRCA1 | | | AGG AAG CTT C | GGG AT |
| ex. 20 | | | | |
| 5382 ins C | | | | |
| Pair 2 for | B1-5382INSCI2 | identification | TGA CGT GTC TGC | ACC TTT CTG TCC TGG |
| BRCA1 | | | TCC ACT TC | GGA TT |
| ex. 20 | | | | |
| 5382 ins C | | | | |
| Pair 3 for | B1-5382INSCK1 | control | CAC TTC CAT TGA | CAA AGG GGA GTG GAA |
| BRCA1 | | | AGG AAG CTT C | TAC AG |
| ex.20 | | | | |
| 5382 ins C | | | | |
| Pair 4 for | B1-5382INSCK2 | control | ATA TGA CGT GTC | CAA AGG GGA GTG GAA |
| BRCA1 | | | TGC TCC AC | TAC AG |
| ex. 20 | | | | |
| 5382 ins C | | | | |
| Pair 1 for | B1EX5IK1 | identification/ | CTC TTA AGG GCA | TTC CTA CTG TGG TTG |
| BRCA1 | | control | GTT GTG AG | CTT CC |
| ex. 5 | | | | |
| 300T→G | | | | |
| Pair 2 for | B1EX5IK2 | identification/ | ATG GCT CTT AAG | TGT GGT TGC TTC CAA |
| BRCA1 | | control | GGC AGT TG | CCT AG |
| ex. 5 | | | | |
| 300T→G | | | | |
| Pair 1 for | B1_4154DELAI | identification | CAA AGG CAT CTC | CAA GCC CGT TCC TCT |
| BRCA1 | | | AGG AAC ATC | TTC TCA |
| ex. 111 | | | | |
| 153 delA | | | | |
| Pair 2 for | B1_4154DELAI | identification | TTG GCT CAG GGT | AAG CCC GTT CCT CTT |
| BRCA1 | | | TAC CGA AG | TGT CA |
| ex. 112 | | | | |
| 4153 delA | | | | |
| Pair 3 for | B1_4154DELAK | control | TTG GCT CAG GGT | GTG CTC CCC AAA AGC |
| BRCA1 | | | TAC CGA AG | ATA AAC |
| ex. 111 | | | | |
| 4154 delA | | | | |
| Pair 4 for | B1_4154DELAK | control | TCC TAG CCC TTT CAC | GTG CTC CCC AAA AGC |
| BRCA1 | | | CCA TAC A | ATA AAC |
| ex. 112 | | | | |
| 4153 delA | | | | |

TABLE 2
Primer sets for analysis of BRCA1 mutations with multiplex PCR.
Primer sets

| | |
|---|---|
| 1 | B1EX5IK1F, B1EX5IK1R, B1_4154DELAI2F, |
| | B1_4154DELAI1R, B1-5382INSCI1F, B1-5382INSCI1R |
| 2 | B1EX5IK2F, B1EX5IK2R, B1_4154DELAK2F, |
| | B1_4154DELAI2R, B1-5382INSCK2F, B1-5382INSCI2R |
| 3 | B1EX5IK1F, B1EX5IK2R, B1_4154DELAI2F, |
| | B1_4154DELAI2R, B1-5382INSCI2F, B1-5382INSCI1R |

In order to achieve comparable amounts of amplified PCR products it is in some cases convenient to optimize the applied proportions of primers. Such optimization depends on several factors, e.g. type and activity of polymerase used or the length and composition of the amplified oligonucleotides, and can be achieved based on publicly available laboratory knowledge. Other components for the diagnostic set, besides the primers, include nucleotides, thermostable polymerase and buffer for the polymerase reaction, that are necessary elements in the mixture of substances for the PCR reaction.

DNA is isolated from peripheral blood leukocytes by conventional methods, and then used as the matrix for the PCR reaction. Conventional diagnostic tests for mutations in the BRCA1 gene, adjusted for the Polish population, are based on multiplex ASO-PCR (mutations 4153delA and 5382insC) and RFLP (mutation C61G) methods.

The reaction mixture recommended for the aforementioned diagnostic test includes a mixture of primers responsible for amplification of a fragment of exon 5 enclosing the location of the eventual mutation C61G. Additional PCR products are indicators for the quality of the PCR reaction and serve as internal controls. Restriction enzyme Avail cuts the PCR product of exon 5 into two smaller fragments, whenever mutation C61G is present, amplification of a fragment of exon 11 only in case mutation 4153delA is present in the analyzed material, amplification of a fragment of exon 20 only in case mutation 5382insC is present in the analyzed material, where the lengths of the PCR products for exons 5, 11 and 20 are chosen to allow for simple and unequivocal identification using electrophoresis in agarose gel.

Here, the reaction ASO-PCR was carried out in an automatic thermocycler (DNA ThermalCycler 9600-Perkin Elmer). The mixture of substances for 25 μl volume comprised: 1 μl (50 ng-200 ng) genomic DNA, 2.5 μl reaction buffer (100 mM Tris-HCl, 500 mM KCL, 15 mM MgCl₂, 1 mg/ml gelatin; pH 8.6), 2-14 μM of each primer, 200 μM of each deoxynucleotide (dATP, dCTP, dGTP and dTTP) and 1 U Taq DNA polymerase. For each reaction there are additionally 3 positive controls (control DNA from carriers of the mutations 5382insC, C16G and 4153delA) and 2 negative controls (control DNA from non-carriers and a control with no DNA at all).

Amplification takes place under the following conditions:
DNA denaturation at 95° C. during 5 minutes
10 cycles consisting each of
   denaturation at 94° C. during 30 seconds
   primer binding at 68-58° C. during 30 seconds* *—for the first 10 cycles the temperature for primer binding is decreased in 1.2° C. for each following cycle (in the first cycle it took 68° C., in the second 66.8° C., in the third 65.6° C., in the fourth 64.4° C., in the fifth 63.2° C., in the sixth 62° C., in the seventh 60.8° C., in the eight 59.6° C., in the ninth 58.4° C. and in the tenth 57.2° C.).
   elongation of complementary DNA at 72° C. during 35 seconds
30 cycles consisting each of
   denaturation at 94° C. during 30 seconds
   primer binding at 57° C. during 30 seconds
   elongation of complementary DNA at 72° C. during 30 seconds

5 μl of PCR reaction products were mixed with 10111 Stop buffer (Solution of saccharose stained with bromophenol blue) and subjected to electrophoresis in agarose gel (1.5% agarose SeaKem FMC, 1× buffer TBE, 25 μg/m| ethidium bromide) under 6V/cm for 30 min. The separated products in the gel were visualized with UV illumination.

### Treatment

Cisplatin chemotherapy was administered at a dose of 75 mg/m² every three weeks for four cycles. Each cycle was 21 days. Granulocyte colony-stimulating factor (G-CSF) was not given to any patient. Dexamethasone (8 mg) was administered once daily for three days after chemotherapy, Ondansetron (Zophran™; GlaxoWellcome) was used for anti-nausea prophylaxis. Toxicity (nausea, vomiting, neutropenia) was assessed using the National Cancer Institute Common Toxicity Criteria after each cycle.

After cisplatin chemotherapy, all patients were treated with mastectomy and axillary lymph node dissection. Surgery was followed by post-operative chemotherapy (four cycles of Adriamycin and Cyclophosphamide). Two patients received radiotherapy. No patient received Trastuzumab (Herceptin™) or hormonal therapy.

### Response Criteria

Clinical response was considered complete if there was no evidence of the primary breast tumor according to the World Health Organization (WHO) criteria (total disappearance of the tumor in breast and lymph nodes). Clinical response was considered ‘partial’ if there was a reduction of ≧50% in the product of the two largest perpendicular diameters of the tumor. If the tumor area showed a reduction of <50% or an increase of >25% in the products of the largest diameter, then the patient as classified as a non-responder. The primary endpoint of this study was pathologic complete response. Pathologic response was considered complete if there was no evidence of invasive breast tumor and the lymph nodes were negative. Pathology specimens were reviewed by two pathologists. If there was evidence of breast carcinoma in situ, but no evidence of invasive disease, this was still considered to be a pathologic complete response.

### Results and Conclusions

From December 2006 to December 2007, 10 patients were enrolled in the study. Patient characteristics are summarized in Table 3. All patients had a BRCA1 mutation; of these, eight (80%) carried the common 5382insC founder mutation. The median age of diagnosis was 45 years (range 38-57 years). The median tumor size before treatment was 2.5 cm (range 1.0-6.5 cm). Clinically-positive lymph nodes were noted in three patients (30%). Nine patients had immunohistochemical studies; all were negative for estrogen receptor, progesterone receptor and ERBB2 (i.e. they were triple-negative). Seven patients received the planned four cycles of cisplatin. Three patients stopped treatment after two cycles. One patient stopped due to side effects (nausea and vomiting) and two patients stopped in order to expedite surgery. No patient experienced febrile neutropenia and no patient received G-CSF. No grade 3 or 4 anemia was observed.

After neoadjuvant cisplatin, no patient had clinical evidence of disease in the breast, but one patient had palpable lymph nodes (clinical response rate 90%). The patient with a partial clinical response had received only two cycles of chemotherapy. All ten patients then had a mastectomy and lymph node dissection. Nine patients achieved a complete pathological response (90%) with no residual disease in the breast or the axilla. The tenth patient (who did not achieve a clinical complete response) had no residual disease in the breast, but three (of 11) axillary nodes were positive for tumor cells. There was no evidence of ductal carcinoma in situ in any breast specimen. The summary of the therapy response can be found in table 4.

This study indicates that a high proportion of women with BRCA1-associated breast cancers will respond to platinum-based chemotherapy. Four cycles of cisplatin in the neo-adjuvant setting resulted in a complete pathologic response in all seven patients who completed treatment. Only one patient did not achieve a pathologic complete response—she completed only two cycles of chemotherapy (of the three patients who received two cycles, two achieved pathologic complete response). Compliance was excellent and only one patient experienced significant toxicity, namely nausea and vomiting (a known side effect of cisplatin).

Despite the small sample size, this is the highest observed rate of pathological complete response for breast cancer reported to date. Initial studies with neoadjuvant therapy with anthracycline and taxane combination chemotherapies achieved pathologic complete response (pCR) rates of 15-34%. With the introduction of trastuzumab (Herceptin™) for HER2 positive patients, much higher pCR rates have been observed (Peintinger et al. Ann. Oncol. 2008, 19(12):2020-5).

Pathologic complete response is a good predictor of recurrence-free survival. Liedtke et al. reviewed outcomes in 1.118 patients who received neo-adjuvant chemotherapy (various agents) at MD Anderson Cancer Center from 1985 to 2004. Overall, 15% of the patients experienced a pathologic complete response, including 57 of 255 patients (22%) with triple-negative breast cancer. Of the 57 patients with triple negative breast cancer who experienced a pathologic complete response, the overall five-year survival rate was 94%, and all deaths occurred in the three years following of treatment. The majority of BRCA1-associated breast cancers are triple-negative. In our study, all patients had triple-negative breast cancer (one was not tested). It is hoped that the survival experience of BRCA1-positive, triple-negative breast cancer patients with pathologic complete response to cisplatin will be as good as that of the patients in the MD Anderson series. This will be the subject of further studies.

Platinum-based chemotherapy is the standard of care for patients with invasive ovarian cancer. Observational trials suggest that carriers of BRCA1 mutations with ovarian cancer may be more sensitive to the effect of platinum-based chemotherapy than non-carrier women (Cass et al, Cancer 2003; 97:2187-95). Our study suggests that the clinical studies of the potential benefit of cisplatin might also be extended to BRCA1 patients with breast cancer and potentially to carriers with other solid tumors, such as prostate and pancreatic cancer. It may be that the best use of platinum-based chemotherapy will be in combination with other agents that increase the effects of DNA-damaging agents, such as the inhibition of enzymes involved in base-excision repair, e.g Poly(ADP-Ribose) polymerase (PARP). The present study confirms the potential benefit of tailoring therapy to specific subgroups of breast cancer patients.

TABLE 3
Baseline clinical characteristics of the study population (N = 10)

| Characteristic | No. | % |
|---|---|---|
| Age, years | | |
| Mean | 46.7 | |
| Range | 38-57 | |
| Type of BRCA1 mutation | | |
| 5382insC | 8 | 80% |
| C61G | 2 | 20% |
| Treating Hospital | | |
| Szczecin | 8 | 80% |
| Bielsko-Biala | 2 | 20% |
| Clinical Tumor Stage | | |
| T1 (<2 cm) | 5 | 50% |
| T2 (≧2 cm to ≧5 cm) | 4 | 40% |
| T3 (>5 cm) | 0 | 0% |
| T4 | 1 | 10% |
| Clinical Nodal Status | | |
| N0 | 7 | 70% |
| N1 | 1 | 10% |
| N2 | 2 | 20% |
| N3 | 0 | 0% |
| Bloom Richardson Grade | | |
| I | 0 | 0% |
| II | 0 | 0% |
| III | 9 | 90% |
| Unknown | 1 | 10% |
| Estrogen Receptor Levels | | |
| Positive | 9 | 90% |
| Negative | 0 | 0% |
| Missing | 1 | 10% |
| Progesterone Receptor Levels | | |
| Positive | 9 | 90% |
| Negative | 0 | 0% |
| Missing | 1 | 10% |
| HER2 Status | | |
| 0 | 9 | 90% |
| 1+ | 0 | 0% |
| 2+ | 0 | 0% |
| 3+ | 0 | 0% |
| Missing | 1 | 10% |

TABLE 4
Response to treatment

| Response | No. | % |
|---|---|---|
| Clinical Response | | |
| Complete Response | 9 | 90% |
| Partial Response | 1 | 10% |
| No Change | 0 | 0% |
| Progressive Disease | 0 | 0% |
| Pathologic Response | | |
| Complete Pathologic Response | 9 | 90% |
| Partial Reseponse | 1 | 10% |
| No Response | 0 | 0% |
| Residual Disease in Breast | | |
| none | 10 | 100% |
| <1 cm | 0 | 0% |
| 1-3 cm | 0 | 0% |
| 4-9 cm | 0 | 0% |
| >9 cm | 0 | 0% |
| Number of Lymph Nodes Positive | | |
| 0 | 9 | 90% |
| 1-3 | 1 | 10% |
| 4-9 | 0 | 0% |
| >9 | 0 | 0% |

### EXAMPLE 2

Cisplatin Chemotherapy of Patients Carriers of a BRCA1 Mutation with Disseminated Breast and Ovarian Cancers

Breast and ovarian cancer are tightly associated cancer types. It is assumed that their genetic background bases on the disruption of the same metabolic pathways. This point of view is supported by the fact that ovarian and breast cancer often appear in familial aggregations, in what is called hereditary breast and ovarian cancer (HBOC) families. Moreover, several genetic alterations are known to increase the risk of both cancer types, for example BRCA2, CHEK2, and of course BRCA1 (Edlich et al. J. Long Term Eff. Med. Implants 2005, 15(5):533-45; Szymanska-Pastemak et al., Gynecol. Oncol. 2006, 102(3):429-31; Cybulski et al. Am. J. Hum. Genet. 2004, 75(6):1131-5).

Given the increased responsiveness of breast cancer patients carriers of a germinal mutation of BRCA1 towards cisplatin chemotherapy (see example 1), the question arises wherever the same effect can be found among ovarian cancer patients carriers of a BRCA1 mutation, and more particularly if this effect is still measurable among the cases with the worst prognosis, i.e. patients with disseminated disease (presence of at least two distant metastasis). The importance of the latter derives from the fact that in most of those cases the cancer is inoperable and the patient's chances rely mostly on chemo- and/or radiotherapy. Choosing directly the correct therapy is particularly critical in those cases.

### Study Design and Eligibility Criteria

The patients selected for this study were retrospectively screened from the database of the oncology department at the Pomeranian Academy of Medicine, Szczecin (Poland). All patients had a histologically or cytologically proven diagnosis of breast cancer or ovarian cancer with evidence of disseminated disease (at least two distant metastasis). All cases were documented for the presence of a BRCA1 constitutional mutation among the dominating founder mutations in Poland (C610, 4153delA and 5382insC) determined by the same techniques described in Example 1. The tumor size had to be measurable per RECIST: X-Ray or physical examination ≧20 mm, and/or spiral CT scan≧10 mm, and/or conventional CT scan≧20 mm. Only individuals were chosen with a life expectancy a priori of at least 3 months, to exclude a technical blur due to extreme disease progression in terminal cancer cases. The maximal ECOG performance status admitted was less than or equal to 2. The definition of minimum adequacy of organ function required prior to study entry were as follows: Absolute neutrophil count (ANC)>1,500/mcl, Platelets>100,000/mcl, Haemoglobin >8 g/dl, Total Bilirubin <1.5×ULN, AST (SGOT)/ALT (SGPT)<2.5×ULN and Creatinine within the normal institutional limits. All patients signed their willingness and ability to comply with scheduled visits, treatment plans, laboratory tests, and other study procedures. A total of 15 patients matched the selection criteria with ages at diagnosis ranging from 32 to 70 years and mean age at diagnosis of 48 years.

Ovarian cancer patients were selected following the same selection criteria. Four individuals matched the criteria with ages at diagnosis ranging from 42 to 45 years and mean age at diagnosis of 43.5 years.

Several additional factors, like age at diagnosis, ER, Her2 and PR receptor status, menopausal status, presence of prior or additional adjuvant therapy different than cisplatin, the degree of compliance with the scheduled dosage and presence of adverse effects, among others.

The response assessment was based on a complete tumor measurement form (the form is carried forward continuously from cycle to cycle). Possible responses were complete response, partial response, stable disease and progressive disease, according to international oncology standards.

### Results and Conclusions

The spectrum of BRCA1 mutations among the breast cancer patients showed a dominance of the mutations C61G and 5382insC (7 cases each), whereas 4153delA was found only in 1 case. Two ovarian cancer patients were carriers of the 5382insC mutation and 1 patient was carrier of the missense mutation C61G.

Some patients were affected by minor adverse effects of the therapy, namely neutropenia (6 cases), nausea (2 cases) and anemia (1 case). Six breast cancer patients received cisplatin as first line of therapy, while nine patients received it as a secondary therapy. For the ovarian cancer patients no information on additional therapy was available.

The response to the therapy in breast cancer patients with disseminated disease was, in general, very positive: seven complete remissions, three partial responses, one case of disease stabilization and just four cases with further disease progression.

Even better among the ovarian cancer patients, three showed a complete remission and one case showed a disease stabilization.

The data are summarized in table 1.

TABLE 1
Response to cisplatin chemotherapy in disseminated breast
and ovarian cancer patients carriers of BRCA1 mutations.

| Type of | No of | Response | | | |
|---|---|---|---|---|---|
| cancer | patients | CR | PR | SD | No response |
| BREAST | 15 | 7 | 3 | 1 | 4 |
| OVARY | 4 | 3 | 0 | 1 | 0 |
| | 19 | 10 (53%) | 3 (16%) | 2 (10%) | 4 (21%) |

(CR: complete remission; PR: partial response; SD: stable disease)

When analyzing disseminated breast and ovarian cancer together, 79% of the patients were responsive to the therapy versus 21% who were not responsive. Considering that disseminated cancer has in general a very negative prognosis the result is completely unexpected. This result strongly supports the hypothesis that cisplatin should be applied as first line chemotherapy or adjuvant chemotherapy in those types of cancer, even in a disseminated status (i.e. presence of several distant metastasis), but specifically only in those cases carriers of a germline BRCA1 mutation. This opens a completely new strategy in the clinical practice to prescribe the most effective therapy in cancer patients.

### EXAMPLE 3

Cisplatin Chemotherapy of Patients Carriers of a BRCA1 Mutation Affected by Cancer at Other Sites Different than the Breast and the Ovaries.

It has been shown in literature that cancer of different types may be genetically associated with a risk increase due to the same polymorphisms (Cybulski et al., Am J Hum Genet. 2004, 75(6):1131-5). BRCA1 is not an exception (Cybulski et al., Eur. J. Cancer Prev. 2008, 17(1):62-6; Maehle et al. Clin. Cancer Res. 2008, 14(22):7569-73; Reguart et al. Clin. Lung Cancer 2008, 9(6):331-9; Easton et al., Lancet 1994, 344(8924):761). A potential common link between BRCA1 genetic profile and chemotherapy outcome was the background of this study. After the evident success applying platin-based chemotherapy on breast and ovary cancer patients carriers of a mutant BRCA1 genotype, it was attempted to identify other types of cancer that could reveal a similar outcome, given the BRCA1 profile of the patient.

### Study Design and Eligibility Criteria

The database of the oncology department at the Pomeranian Academy of Medicine, Szczecin (Poland), was retrospectively screened for patients affected by cancer types different than the breast or ovary (histologically or cytologically proven) since those cancer types had already been analyzed in Examples 1 and 2. Those patients had to be carriers of at least one constitutional mutation of the gene BRCA1, among the dominating founder mutations in Poland (CMG, 4153delA and 5382insC). The presence of a mutation was determined by the same techniques described in Example 1. Moreover the patients had to be object of a chemotherapy with cisplatin.

The group that qualified matching all criteria consisted of five patients affected by cancer of the colon (1 subject), the pancreas (1 subject), the gallbladder (1 subject) and the lung (2 subjects). In 60% of the cases (both lung cancer patients and the colon cancer patient) a complete remission was reported after the application of the chemotherapy. The gallbladder cancer patient had a partial remission and the pancreatic cancer patient showed a stabilization of the disease. In none of the cases, there was a lack of response towards cisplatin. The results are summarized in table 1.

TABLE 1
Response to cisplatin chemotherapy in cancer patients
carriers of BRCA1 mutations (retrospective).

| Type of | No of | Response | | | |
|---|---|---|---|---|---|
| cancer | patients | CR | PR | SD | No response |
| Colon | 1 | 1 | | | 0 |
| Lung | 2 | 2 | | | 0 |
| Pancreas | 1 | | | 1 | 0 |
| Gallbladder | 1 | | 1 | | 0 |
| Total | 5 | 3 (60%) | 1 (20%) | 1 (20%) | 0 |

(CR: complete remission; PR: partial response; SD: stable disease)

All patients showed some response to the drug, 80% of them at least a partial remission and 60% even a complete remission. This unexpectedly positive result strongly suggests that the effectiveness of cisplatin is valid not just for breast an ovarian cancers localized (Example 1) or disseminated (Example 2), but moreover in all cancer types, given a positive BRCA1 mutation profile of the patient. This finding is of critical relevance for the future of clinical oncology, since genetic testing prior to chemotherapy prescription is demonstrated to be indispensable to maximize therapy effectiveness.

1. A method for early detection of enhanced clinical response towards DNA damaging chemotherapy in cancer patients, which comprises detecting a germline alteration in the sequence of BRCA1 gene in a biological sample from the analyzed subject, wherein the mutation carrier genotype is indicative of significantly increased clinical response to chemotherapy with DNA damaging drugs in cancer patients.

2. The method of claim 1, wherein examined structural alteration is identified by comparison of the structure of the altered BRCA1 variant with the wild type.

3. The method of claim 1, wherein investigated human subject is a person of, at least partially, known ethnic origin.

4. The method of claim 1, wherein the constitutional mutations of BRCA1 gene being indicative of significantly increased response to chemotherapy with DNA damaging drugs are identified from a set or panel of BRCA1 constitutional mutations, which are characteristic for the ethnic population of the patient.

5. The method of claim 1, wherein investigated human subject is a person of unknown ethnic origin.

6. The method of claim 1, wherein the constitutional mutations of BRCA1 gene being indicative of significantly increased response to chemotherapy with DNA damaging drugs is identified from a set or panel of BRCA1 constitutional mutations, which comprise all known constitutional mutations of BRCA1 or a sample of the most frequent ones.

7. The method of claim 1, wherein the mode of detection of constitutional BRCA1 mutations is based on analysis of DNA, RNA or proteins.

8. The method according to claim 7, wherein DNA or RNA testing is performed using any conventional technique of direct mutation detection, such as sequencing, but more preferably any conventional technique of indirect mutation detection, selected among those such as ASA-, ASO-, RFLP-PCR, Taqman RT-PCR, Maldi-T of or microarray methods, preferably based on constitutional mutation panels.

9. The method according to claim 7, wherein the presence of the polypeptide encoded by the BRCA1 gene with germline alteration is detected with the use of antibodies or other substances specific for this polypeptide or its fragment.

10. The method of claim 1, wherein the DNA damaging drugs conventionally used in pharmacy for cancer chemotherapy are, at least, those derived from platinum, such as cisplatin, carboplatin, triplatin, satraplatin, nedaplatin or oxaliplatin.

11. The method of claim 1, wherein genetic testing is indicated to be performed among all cancer patients, for which chemotherapy with DNA damaging drugs is intended, and particularly favorable for the case of neoadjuvant therapy for which a quick assignment of the correct chemotherapy is of highest critical clinical relevance.

12-15. (canceled)

16. Composition for prediction of increased response to chemotherapy with DNA damaging drugs in cancer patients, comprising at least two different oligonucleotides allowing amplification of region of genome of said human subject containing at least one mutation among a set or panel of BRCA1 constitutional mutations, preferably comprising all constitutional mutations, which are characteristic for the ethnic population of the patient, or other BRCA1 alterations with analogous properties, or sharing a haplotype with the former ones.

17. (canceled)

18. The method of identification of genetic markers being predictive of significantly increased response to chemotherapy with DNA damaging drugs, characterized by comprising the examination of samples containing genomic DNA from patients affected by specific cancer and comparing the frequency of structural change within BRCA1, or regions in linkage disequilibrium, between examined patients and controls from general population, wherein the alteration significantly over represented in cancer patients is then regarded as genetic marker being predictive of significantly increased response to chemotherapy with DNA damaging drugs.
If a telephone interview would be of assistance in advancing prosecution of the subject application, applicants' undersigned attorney invites the Examiner to telephone him at the number provided below.


SEQUENCE LISTING (20 sequences)

SEQ ID NO: 1 - DNA, 22 bp, artificial
  primer B1-5382INSCI1_F
cacttccattgaaggaagcttc

SEQ ID NO: 2 - DNA, 20 bp, artificial
  primer B1-5382INSCI1_R
tacctttctgtcctggggat

SEQ ID NO: 3 - DNA, 20 bp, artificial
  primer B1-5382INSCI2_F
tgacgtgtctgctccacttc

SEQ ID NO: 4 - DNA, 20 bp, artificial
  primer B1-5382INSCI2_R
acctttctgtcctggggatt

SEQ ID NO: 5 - DNA, 22 bp, artificial
  primer B1-5382INSCK1_F
cacttccattgaaggaagcttc

SEQ ID NO: 6 - DNA, 20 bp, artificial
  primer B1-5382INSCK1_R
caaaggggagtggaatacag

SEQ ID NO: 7 - DNA, 20 bp, artificial
  primer B1-5382INSCK2_F
atatgacgtgtctgctccac

SEQ ID NO: 8 - DNA, 20 bp, artificial
  primer B1-5382INSCK2_R
caaaggggagtggaatacag

SEQ ID NO: 9 - DNA, 20 bp, artificial
  primer B1EX5IK1_F
ctcttaagggcagttgtgag

SEQ ID NO: 10 - DNA, 20 bp, artificial
  primer B1EX5IK1_R
ttcctactgtggttgcttcc

SEQ ID NO: 11 - DNA, 20 bp, artificial
  primer B1EX5IK2_F
atggctcttaagggcagttg

SEQ ID NO: 12 - DNA, 20 bp, artificial
  primer B1EX5IK2_R
tgtggttgcttccaacctag

SEQ ID NO: 13 - DNA, 21 bp, artificial
  primer B1_4154DELAI1_F
caaaggcatctcaggaacatc

SEQ ID NO: 14 - DNA, 21 bp, artificial
  primer B1_4154DELAI1_R
caagcccgttcctctttctca

SEQ ID NO: 15 - DNA, 20 bp, artificial
  primer B1_4154DELAI2_F
ttggctcagggttaccgaag

SEQ ID NO: 16 - DNA, 20 bp, artificial
  primer B1_4154DELAI2_R
aagcccgttcctctttgtca

SEQ ID NO: 17 - DNA, 20 bp, artificial
  primer B1_4154DELAK1_F
ttggctcagggttaccgaag

SEQ ID NO: 18 - DNA, 21 bp, artificial
  primer B1_4154DELAK1_R
gtgctccccaaaagcataaac

SEQ ID NO: 19 - DNA, 22 bp, artificial
  primer B1_4154DELAK2_F
tcctagccctttcacccataca

SEQ ID NO: 20 - DNA, 21 bp, artificial
  primer B1_4154DELAK2_R
gtgctccccaaaagcataaac